(19) United States
(12) Patent Application Publication
(10) Pub. No.: US 2018/0147004 A1
(43) Pub. Date: May 31, 2018

(54) DUAL MODE ELECTROSURGICAL APPARATUS AND METHOD OF MANUFACTURE

(71) Applicant: Robert E. DeCou, Redwood City, CA (US)

(72) Inventor: Robert E. DeCou, Redwood City, CA (US)

(21) Appl. No.: 15/363,971

(22) Filed: Nov. 29, 2016

(51) Int. Cl. A61B 18/14; B23K 11/11

(52) U.S. Cl. CPC A61B 18/1477; A61B 2018/124; B23K 11/11

(57) ABSTRACT

A monopolar-based electrosurgical handpiece includes a body, a distal tip, and a tip-support housing joining the distal tip to the body. A first electrode located in the distal tip is coupleable to a first active lead for cutting tissue, together with a remote return electrode. The first electrode includes a discrete exposed cutting surface area. The distal tip also includes a second electrode coupleable to the first active lead for coagulating tissue, together with the remote return electrode. The second electrode has a discrete exposed coagulating surface area larger than the discrete exposed cutting surface area. An electrically non-conducting support is secured to the first electrode, and defines the discrete exposed cutting surface area where the first electrode is uncovered by the electrically non-conducting support. The support strengthens the distal tip, and electrically separates the first electrode from the second electrode. The handpiece further includes at least one switch in the body for selectively coupling the first active lead to one of the first electrode and the second electrode.

### CROSS-REFERENCE TO RELATED APPLICATIONS

None.

### BACKGROUND OF THE INVENTION

The invention relates to electrosurgery, and in particular, to monopolar-based electrosurgical handheld devices.

Monopolar electrosurgery is a type of electrosurgery in which current is applied through a handheld active electrode, through the patient, and back to the electrosurgical generator through a return electrode pad.

Monopolar-based electrosurgical devices typically perform two functions during surgery including cutting tissue and coagulating bleeding vessels. Cutting requires high current density, achieved by applying a potential to a small surface area, the small surface area allows for precise control of the electric potential and thus a finer cut. Coagulation requires application of a potential to a larger surface area to quickly control bleeding. Examples of various monopolar-based electrosurgical devices are described in the U.S. Pat. Nos. 3,799,168; 4,545,375; 7,959,633. Some well-known commercial monopolar-based electrosurgical devices are the BOVIE® ESP1 manufactured by Bovie Medical Corporation, (Clearwater, Fla.); the GoldLine® Pencil manufactured by ConMed Corporation (Utica, N.Y.) and the Force TriVerse™ Pencil manufactured by Medtronic (Minneapolis, Minn.).

Monopolar-based electrosurgical devices, however, face a number of challenges such as, e.g., (a) the coagulation electrode needed is much larger than the cutting electrode, (b) both of the coagulation and cutting electrodes need to be integrated into a single working distal end and work seamlessly together during a surgical procedure, and (c) the electrosurgical device must be able to selectively control activation of the coagulation electrode or the cutting electrode.

Accordingly, an improved apparatus that overcomes the challenges described above is desired.

### SUMMARY OF THE INVENTION

A monopolar-based electrosurgical handpiece is operable with a generator and a first active lead and a remote return electrode. Both the first active lead and the remote return electrode are coupled to the generator. The handpiece includes a body, a distal tip, and a tip-support housing joining the distal tip to the body. A first electrode is located in the distal tip, and coupleable to the first active lead for cutting tissue, together with the remote return electrode. The first electrode has a discrete exposed cutting surface area. A second electrode is located in the distal tip, and coupleable to the first active lead for coagulating tissue, together with the remote return electrode. The second electrode has a discrete exposed coagulating surface area larger than the discrete exposed cutting surface area. An electrically non-conducting support is secured to the first electrode, and defines the discrete exposed cutting surface area where the first electrode is uncovered by the electrically non-conducting support, and electrically separates the first electrode from the second electrode. At least one switch is incorporated into the body for selectively coupling the first active lead to one of the first electrode and the second electrode.

In embodiments the first electrode is thin and planar shaped.

In embodiments the second electrode comprises two thin and planar-shaped coagulation members.

In embodiments the two coagulation members sandwich the first electrode therebetween. In embodiments each of the two coagulation members has a thickness ranging from 0.005 to 0.020 inches.

In embodiments the two coagulation members are electrically connected and mechanically affixed to one another through an aperture in the first electrode.

In embodiments the first electrode has a uniform thickness. In embodiments the thickness of the first electrode may range from 0.002 to 0.010 inches.

In embodiments substantially all of the first electrode is covered by the electrically non-conducting body.

In embodiments the electrically non-conducting support is mechanically secured between the first electrode and the second electrode. The mechanical affixation may be carried out by one or more spot welds or fasteners.

In embodiments the electrically non-conducting support is affixed adhesive-free. The electrically non-conducting support may be a material selected from fluoropolymer or ceramic. The thickness of the electrically non-conducting support may range from 0.001 to 0.010 inches.

In embodiments the electrically non-conducting support comprises a plurality of discrete layers.

In embodiments the tip-support housing is detachably joined to the body.

In embodiments the body comprises at least one actuating element to operate the at least one switch selected from the group consisting of a button, lever, knob, slide, and trigger.

In embodiments the distal tip has a hook shape.

In embodiments the apparatus further comprises a third electrode located in the distal tip, and coupleable to the first active lead for coagulating tissue, together with the remote return electrode.

In embodiments different waveforms are delivered to the active electrode from the electrosurgical unit corresponding to coagulation and cutting mode.

In embodiments different power levels are delivered to the active electrode from the electrosurgical unit corresponding to coagulation and cutting mode.

In embodiments the handpiece is operable in dry-field applications. In embodiments an electrically conductive fluid (such as blood or saline) is unnecessary for achieving sufficient cutting and coagulation.

In embodiments a method of coagulating tissue or a vessel includes inserting a distal tip in the treatment area, and applying a coagulation output to a coagulation electrode assembly, and in some embodiments, applying a coagulation output to a cutting and coagulation electrode.

In embodiments a method of manufacturing a monopolar-based electrosurgical handpiece comprises arranging an elongate planar-shaped electrically non-conducting body on each side of an elongate planar-shaped cutting electrode, and an elongate planar-shaped coagulation electrode on top of each elongate planar-shaped electrically non-conducting body such that a cutting surface area of the cutting electrode is left exposed; and mechanically securing each coagulation electrode and electrically non-conducting body to the cutting electrode.

In embodiments the step of mechanically securing is performed by welding.

In embodiments each elongate planar-shaped electrically non-conducting body comprises an opening extending there though.

In embodiments the elongate planar-shaped cutting electrode comprises an aperture extending there though, and wherein each opening in the electrically non-conducting body is aligned with the aperture.

In embodiments the method further comprises positioning within each said opening and aperture an elongate planar-shaped electrically conducting support, and the support electrically connecting each elongate planar-shaped coagulation electrode to form an integrated multi-surface coagulation electrode.

In embodiments the elongate planar-shaped electrically conducting support and elongate planar-shaped coagulation electrode are made of stainless steel.

In embodiments a system includes any one of the handpieces described above in combination with a generator wherein the generator is operable in a cutting mode and a coagulation mode, and a first active lead and a remote return electrode coupled to the generator.

In embodiments the device electrode assembly and insulation are held together solely mechanically and without adhesives or chemicals. In contrast, a number of conventional electrode assemblies are chemically coated and rely on the chemical bonds to maintain function. The chemical bonds can break down during surgery both through mechanical action as well as exposure to high electrical potentials, currents, and temperatures.

In embodiments the working or business end of the device has a much thinner cutting electrode than conventional device assemblies. In embodiments a thin electrode sheet is supported by a larger, structurally robust, pair of coagulation electrodes Advantageously, a higher current density is generated and the assembly achieves a better cutting performance than a thicker cutting electrode.

In embodiments the cutting electrode is not tapered like other conventional devices. In a conventional tapered electrode design, for example, the thin edge wears rearward to a thicker section and consequently, performance degrades. In contrast, as the cutting electrode of the subject invention wears, performance does not degrade. The thickness of the cutting electrode is constant because the planar thin sheet defines the cutting edge's size. The thickness is constant. This advantage is highlighted if and when the cutting electrode is exposed to a coagulation waveform, which is not uncommon and can prematurely wear cutting electrodes.

In embodiments a multi electrode device is configured for use with conventional electrosurgical generators typically present in all hospitals. Additional training and setup is not required to operate the device described herein. Despite the operational limits of a standard ESU, which is incompatible with a multi-electrode device because the ESU cannot steer the output to the proper electrode, the handpiece and implements of the subject invention have a built-in control scheme to steer the output to the proper electrode.

Still other descriptions, objects and advantages of the present invention will become apparent from the detailed description to follow, together with the accompanying drawings.

### BRIEF DESCRIPTION OF THE DRAWINGS

FIG. 1 depicts a surgical procedure on a patient by a physician using an electrosurgical handpiece and generator.

FIG. 2 is an enlarged view of an electrosurgical generator, handpiece, and return electrode.

FIG. 3 is an enlarged view of an electrosurgical handpiece.

FIG. 4A is a perspective view of the tip-support housing shown in FIG. 3 with the handle body removed.

FIG. 4B is a top view of the tip-support housing shown in FIG. 4A.

FIG. 4C is a side view of the tip-support housing shown in FIG. 4B.

FIG. 5A is an enlarged view of a portion of the distal tip shown in FIG. 4B.

FIG. 5B is an exploded view of the distal tip shown in FIG. 4A.

FIGS. 6A-6C are various schematic diagrams of electrical circuits for operating an electrosurgical system in dual modes.

FIG. 7 is a side view of another tip-support housing shown with the handle body removed.

FIG. 8 is a top view of the tip-support housing shown in FIG. 7.

FIG. 9A is a side view of another electrosurgical handpiece having a J-hook shaped distal tip.

FIG. 9B is an enlarged side view of the tip-support housing shown in FIG. 9A.

FIG. 9C is an enlarged view of a portion of the distal tip shown in FIG. 9B.

FIG. 10 is an enlarged view of an alternative variation of the distal tip section shown in FIG. 9C.

FIG. 11A is a side view of another electrosurgical handpiece having a L-hook shaped distal tip.

FIG. 11B is an enlarged side view of the tip-support housing shown in FIG. 11A.

FIG. 11C is an enlarged view of a portion of the distal tip shown in FIG. 11B.

FIG. 12 is an enlarged view of an alternative variation of the distal tip section shown in FIG. 11C.

FIG. 13 is a perspective view of another electrosurgical implement having a distal tip section in a folded arrangement.

FIG. 14 is an exploded view of the distal tip shown in FIG. 13.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. It is also to be appreciated that unless defined otherwise, all technical and scientific terms used herein have the same meaning as commonly understood by one of ordinary skill in the art to which this invention belongs.

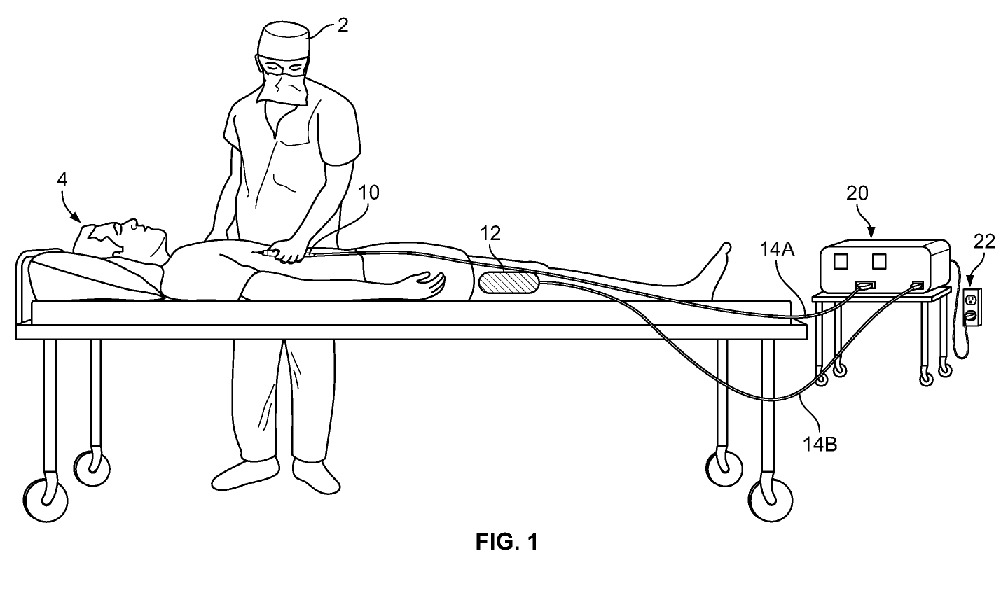

FIG. 1 depicts a physician 2 performing a surgical procedure on a patient 4 using an electrosurgical handpiece 10. Exemplary surgical procedures include, e.g., laparoscopic and arthroscopic procedures. The electrosurgical handpiece 10 and a remote return electrode 12 are shown electrically coupled to a generator 20 by lead cables 14A, 14B, respectively. Lead cables may comprise one or more leads or wire. The generator 20 is coupled to a power source via wall outlet 22.

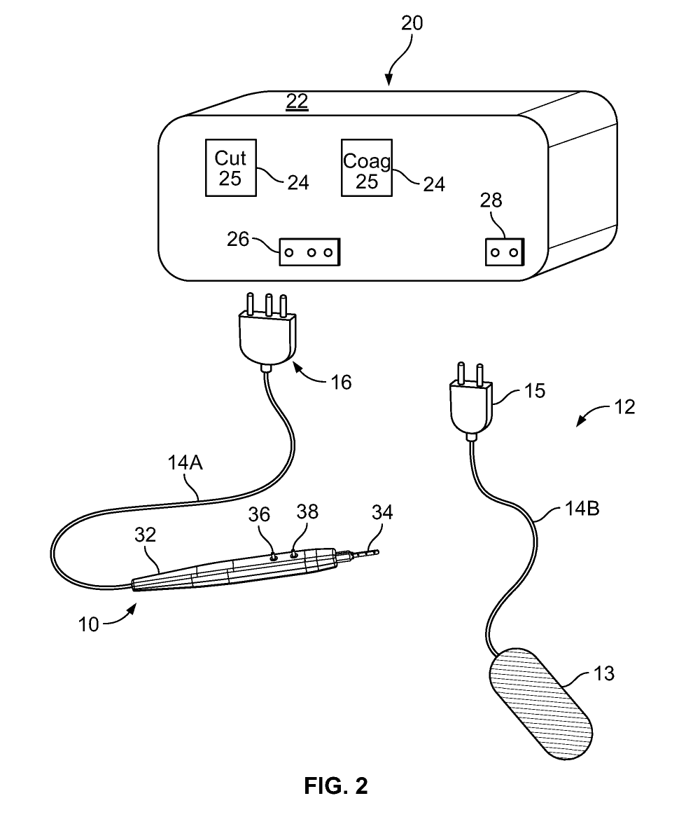

FIG. 2 is an enlarged view of the electrosurgical handpiece 10, return electrode 12, and generator 20 shown in FIG. 1. The generator 20 is shown having a housing 22, displays 24, female device connector 26, and a female return electrode connector 28.

The handpiece 10 is electrically coupleable to the generator with cable or electrical lead cable 14A and male pin connector 16. Handpiece is shown having a body 32, and an electrode blade 34 extending distally therefrom. Several buttons are shown incorporated in the body of the handle to activate the electrosurgical device including a coagulation mode button 36, and a cutting mode button 38. Although the coagulation button is shown proximal the cutting mode button, the arrangement and shape of the buttons may vary widely and take other configurations including levers, knobs, slides, and other means for causing the device to operate as described herein.

The return electrode is shown having a large electrode-like pad 13, lead 14B, and male pin connector 15. With reference again to FIG. 1, the return electrode pad is placed on the patient in a remote location from the electrosurgical blade. Current flows from the selected electrode in the tip of the handpiece, to the remote electrode wherein cutting, ablation, or coagulation results in the vicinity of the electrode tip where the current density is high or a maximum.

In operation, the generator 20 supplies current to the handpiece. The generator may include a coagulation waveform and a cutting waveform, as well as adjustable power levels for each mode. The generator delivers and controls the current based on inputs from the handpiece and generator. The generator may include various components such as a main line input, AC/DC converter, variable DC controller, RF output stage and sub stages to generate the different Cut and Coag outputs, a system to monitor electrical output (voltage, current, power, etc.), feedback control circuitry to adjust the DC supply and the RF output, input switches and controls, A to D converter, and other components. Examples of electrosurgical generators are the Force FX™ manufactured by Medtronic (Minneapolis, Minn.), System 5000™ manufactured by ConMed (Utica, N.Y.), MegaPower® manufactured by MegaDyne (Draper, Utah). Additional generators are described in U.S. Pat. Nos. 4,658,819; 6,830,569; 7,001,381, all of which are incorporated herein by reference in their entirety.

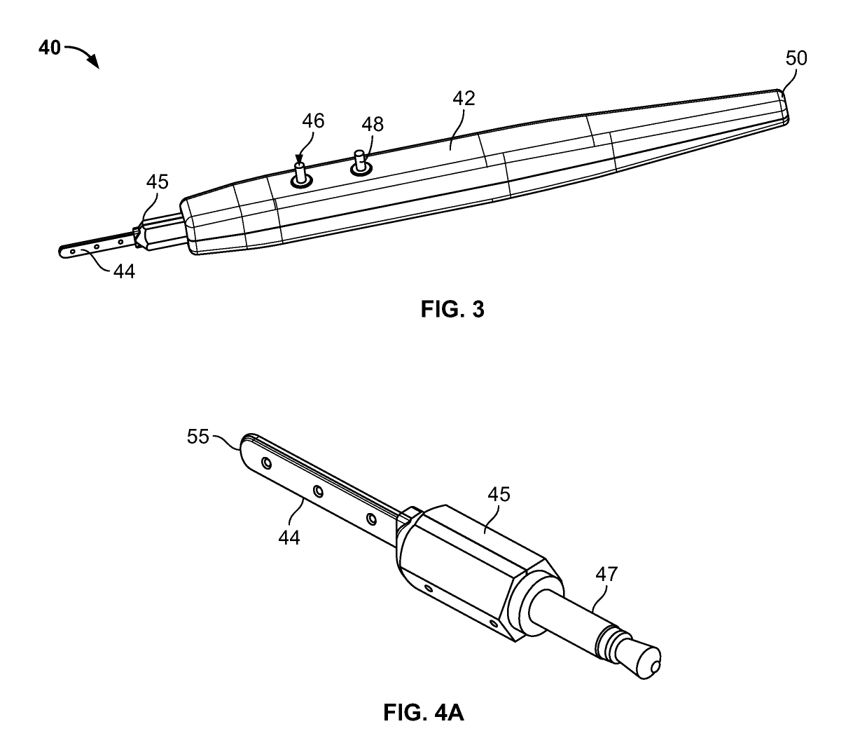

FIG. 3 is an enlarged view of an electrosurgical handpiece 40 similar to that shown in FIG. 2. The handpiece 40 includes a tubular-shaped body 42, an active distal tip 44, a tip-support housing 45, and a tapered proximal end 50 where the electrical lead cable would connect or extend to the generator (not shown). A cut button 46 and coagulation button 48 are shown protruding from the body 42. The shape and arrangement of the components collectively facilitate single hand operation including selectively activating the coagulation or cutting mode as will be described herein.

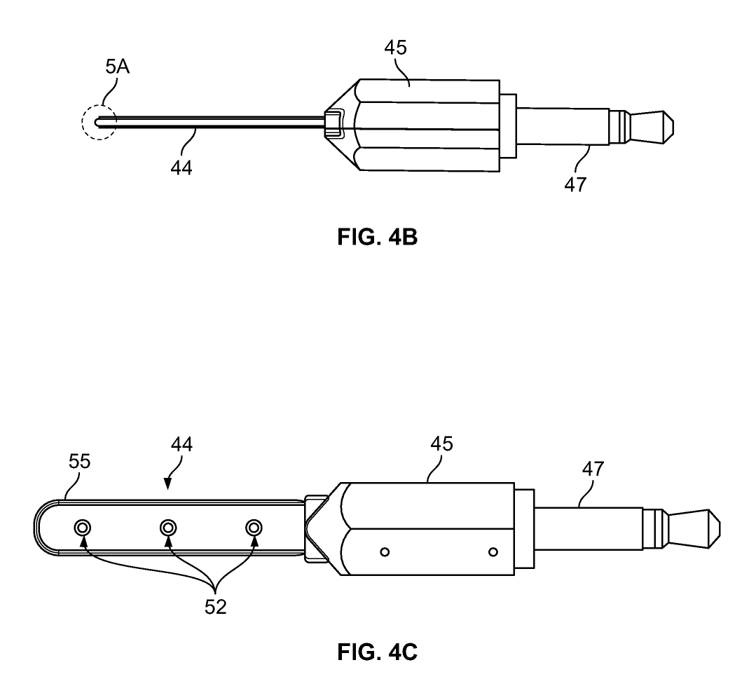

FIGS. 4A-4C are various views of the distal tip 44 and tip-support housing 45 shown in FIG. 3 with the handle body 42 removed. The distal tip 44 is shown having a planar rectangular shape and a curved or semi-circular distal most portion 55.

An electrically conductive male connector in the form of a pin 47 is shown extending proximally from the tip-support housing 45. A female connector in the distal end of the body 42 receives the pin 47 to pass current to and from the generator to the tip-support housing and distal tip. In embodiments, the tip-support housing is detachable from the body 42. For example, the tip-support housing and handle may connect with one another by snap fit, press fit, interference fit, deflecting tabs, or other means. In embodiments, the tip-support housing has a hexagonal shape cross section which allows for keying of the tip relative to the handle.

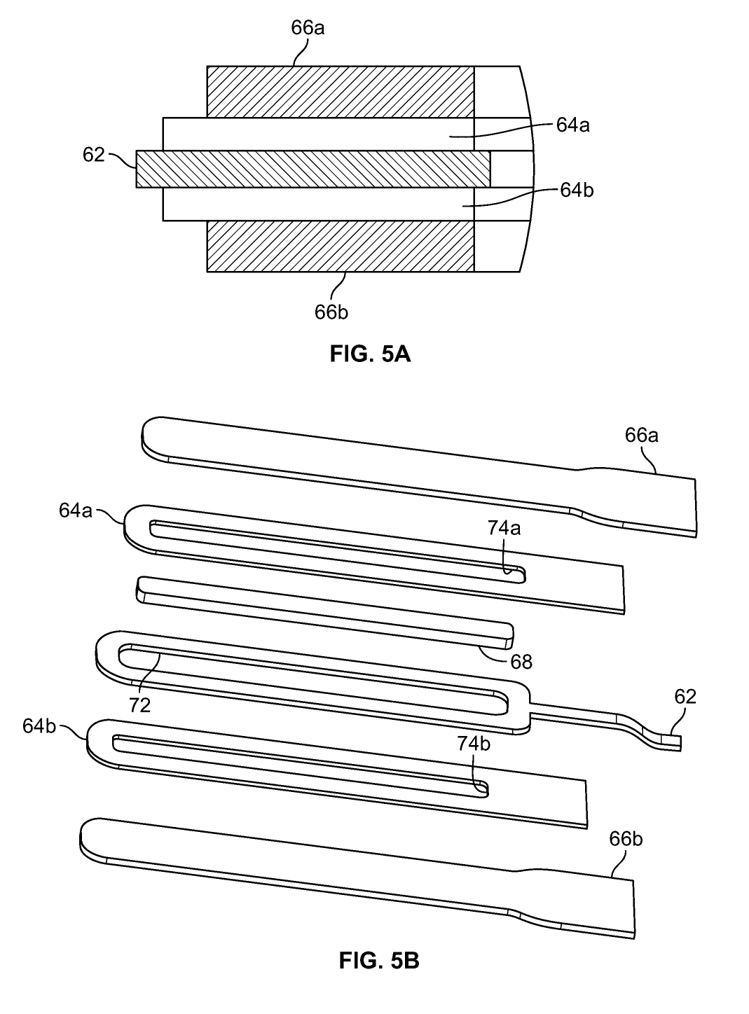

FIG. 5A is an enlarged view of a portion of the distal tip 44 shown in FIG. 4B. The distal tip 44 is shown having a thin, sheet-like cutting electrode 62, insulation layers 64a, 64b, and a two-component larger electrode 66a, 66b intended for coagulating. The insulation layers 64a, 64b cover the cutting electrode 62 and are held in place by the two larger, thicker coagulation electrodes 66a, 66b. The insulation layers 64a, 64b are connected to one another as described further herein. The configuration shown in FIG. 5A creates a sandwich-like composite structure that has two functional independent and electrically isolated electrodes: one functional unit for cutting 62 and one larger surface area functional unit for coagulation 66a,66b.

In embodiments the cutting electrode 62 is relatively thin, having a thickness in the range of 0.002 to 0.005 inches. Exemplary materials for the cutting electrode include tungsten.

In embodiments the coagulation electrode 66 is relatively thick compared to the cutting electrode 62. Each coagulation member 66a,66b may have a thickness of about 0.01 inches.

With reference to FIG. 5A described above, and FIG. 5B which is an exploded view of the distal tip 44 shown in FIG. 4A, the cutting electrode 62 is supported and covered by insulating layers 64a, 64b such that the relatively large lateral sides of the electrode 62 are substantially covered, which results or defines a discrete cutting surface area (namely, the edge and a very small portion of the lateral surfaces of the cutting electrode 62) being exposed to the tissue.

In an exemplary embodiment the exposed cutting area is approximately 0.03 square inches while the approximate coagulation area is 0.13 squares inches. This gives a ratio of approximately at least 4:1 coag to cutting surface area. However, the ratio of the coag area to cutting area may vary. In another embodiment, the exposed cutting area is smaller, the ratio may be as high as approximately 24:1 coag to cutting surface area.

The support layers 64a, 64b are electrically non-conducting and insulating. Support layers 64a,64b along with layers 66a, 66b also serve to strengthen the distal tip. Support layers increase the yield strength of the assembly to a value in the range between 30,000 to 140,000 lbs./sq. inch. The supports thus shore up the thin or even delicate inner cutting electrode.

An exemplary material for the support layers is a fluoropolymer sheet such as, for example, a 0.007 inch PFA (Perfluoroalkoxy alkane) sheet. The insulation layers 64a, 64b are secured against the electrode 62. In embodiments, the insulation layers are mechanically secured to the cutting electrode without adhesives, chemical bonds, or fusion.

In embodiments, thicker sheets of metal 66a, 66b are welded (e.g., spot welds 52) to one another via intermediary electrically-conducting piece 68. Aperture 72 in the cutting electrode and apertures 74a, 74b in the insulation create a passage through which the outer coagulation members 66a, 66b are electrically coupled to one another. The outer members 66a, 66b hold the blade 62 and insulation 64a, 64b fixed in place and also collectively serve as the large (surface area) coagulation electrode.

Intermediary electrically-conducting piece 68 serves to conduct electricity from one coag plate 66a to another coag plate 66b. Intermediary electrically-conducting piece 68 may also serve to hold the assembly together. For example, if the intermediary piece 68 is made of a plastically deformable material and deformed (e.g., during the spot welding process), the internal stresses formed by the deformation can aid in holding the assembly together after the device is built. Examples of suitable materials for the intermediary piece 68 include electrically conducting and plastically deformable materials such as, without limitation, stainless steel, copper or aluminum.

The combination of layers described above including the presence of support layers, and thicker outer coagulation electrode members, strengthen the distal tip, making it more robust during operation than conventional electrosurgical fine cutting-type devices.

Embodiments described herein can generate sufficiently high current density near the thin cutting electrode edge to cleanly, sharply, accurately, quickly and easily cut tissues. Additionally, embodiments described herein can do so while using the “pure” cut mode present on a number of conventional electrosurgical generators at settings of approximately 10 to 50 Watts. A custom generator is not required to operate the handpiece described herein in various embodiments.

The coagulation electrode assembly 66a, 66b operates in the same power domain as the cutting electrode approximately 10 to 50 Watts, however, it uses the coagulation mode of the ESU which operates at higher voltages and lower duty cycles than the cut mode. A non-limiting exemplary duty cycle is 6%, or in embodiments, may range from 5-8%. As described herein, embodiments of the invention can deliver and steer the output as needed to the desired electrode or electrode array, thus avoiding excessive wear on the unintended electrode.

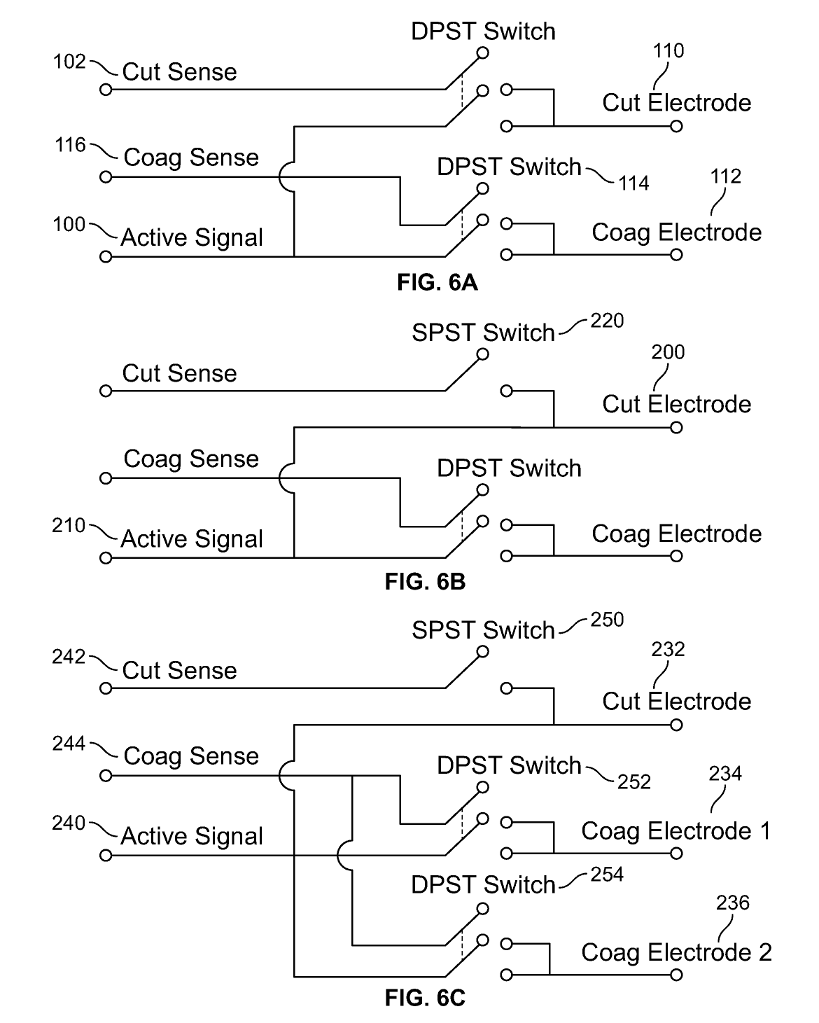

FIGS. 6A-6C are various schematic diagrams of electrical circuits for operating a dual mode electrosurgical handpiece with a monopolar electrosurgical generator. Two types of output are delivered to the activated electrode in the handpiece: a cutting signal and coagulation signal.

In embodiments, a user presses a button that connects the activated electrode (cut or coag electrode) to the output signal, and simultaneously, connects a sense terminal on the generator that corresponds to the requested output signal (Cut or Coagulation signal). Once the connection is made, the requested output is then delivered or steered to the proper electrode on the device.

FIG. 6A illustrates one embodiment configured to electrically connect the Cut sense terminal 102 and active output connection 100 to the cutting electrode 110 when the Cut signal is called. The coagulation electrode 112 floats (i.e., is not connected to the generator).

When the physician desires coagulation, and presses the coagulation button, a double pole single throw (DPST) switch 114 connects the active output connection 100 to both the coagulation electrode (or array) 112 and the Coagulation sense terminal 116. When the switch 114 is released, the connection is broken and the coagulation electrode (or array) electrically floats (i.e., is not connected to either the active output connection or the Coagulation sense terminal).

FIG. 6B illustrates another control scheme configuration. The control scheme described in FIG. 6B is similar to the control scheme described in FIG. 6A except that the cutting electrode 200 is continuously connected to the active output connection 210 and is activated by a single-pole, single-throw (SPST) switch 220 to electrically couple the Cut sense terminal to the active signal. During Coagulation, the cutting electrode need not be electrically isolated from the Coagulation output because coagulation does not require the high current density be isolated to a small area to perform a fine, thin cut. Rather Coagulation requires a large surface area to allow the quick application of the Coagulation effect to the bleeding tissues, including the large areas of exposed tissues that were formed by the cut. The device will operate properly during coagulation even though both types of electrodes are active. Unlike prior art electrosurgical handpieces in which the cutting electrode tends to suffer excessive wear to the extent it is inoperable after minimal use, the wear on the cutting electrode assembly described in embodiments of the present invention is mitigated by the arrangement and structures described herein.

FIG. 6C is another schematic diagram of an electrical circuit for operating a dual mode electrosurgical handpiece with a monopolar electrosurgical generator. The diagram shown in FIG. 6C is an embodiment for separately controlling multiple coagulation and cutting electrodes.

The circuit shown in FIG. 6C includes three electrodes including cut electrode 232, first coag electrode 234, and second coag electrode 236. Both coagulation electrodes use double pole, single-throw (DPST) switches 252, 254 for activation.

Cut electrode 232 is shown using single pole single throw (SPST) switch 250. Cutting is activated by pressing switch 250 to connect the Cut Sense with the active signal. Because the coag electrodes are left floating when they are not active, the handle circuit effectively steers the output to the proper electrode, namely the cut electrode 232.

Coagulation is carried out by directing the active signal to the first coag electrode 234 or second coag electrode 236 by pressing switch 252 or switch 254, respectively. In the above described manner, any one of three different electrodes can be activated by the handle.

Alternatively, an electrosurgical device could have two cutting electrodes and one coagulation electrode. Indeed, the design allows for selectively controlling (e.g., steering) the output to any one or more electrodes.

In embodiments, a control circuit is incorporated into the handpiece and only one active lead from the generator is coupled to both the cut and coagulation electrode. Buttons, levers, knobs, slides, or other elements actuate the switches described herein to steer the output to the desired electrode.

Alternatively, in other embodiments not shown, each types of electrode has a designated electrical lead from the generator. For example, a first lead is coupled to the cutting electrode and a second lead is connected to a coag electrode. The generator includes circuitry to selectively activates the proper electrode as requested by the user. Indeed, the generators, handpieces, and controllers may vary widely. The invention is intended only to be limited as recited in the appended claims.

### Alternative Embodiments

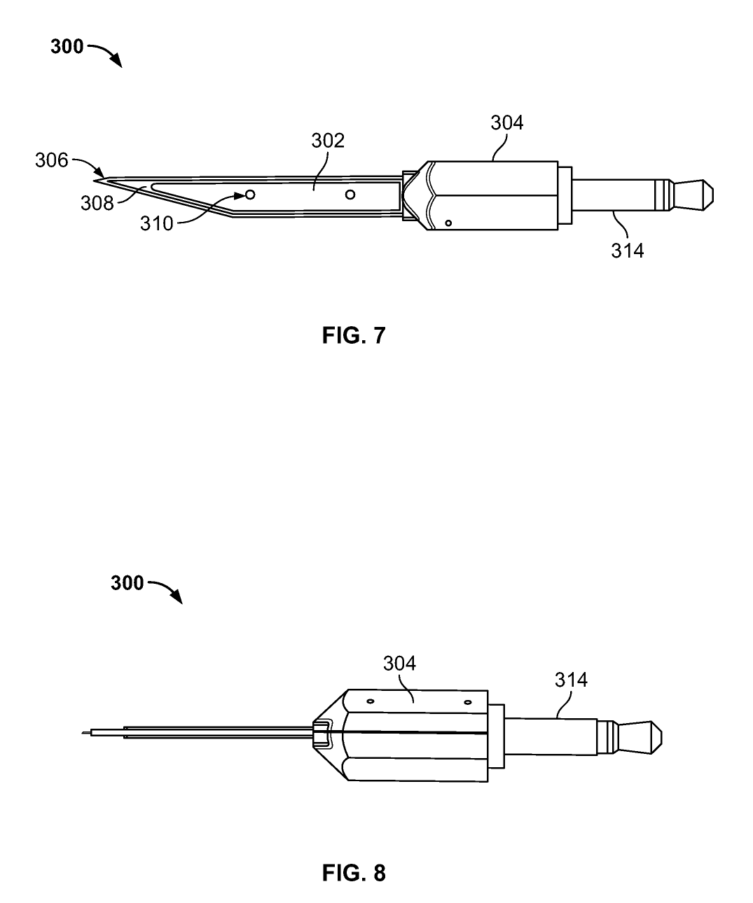

FIGS. 7-12 depict various alternative shapes for the distal tip.

With reference to FIGS. 7-8, an electrosurgical implement includes a scalpel-like shaped blade 300 extending from a tip-support housing 304, and pin connection 314. The shape of blade may be designed to match any number of scalpel shapes including without limitation #10, #11, #12 or #15 model scalpel blades. A coagulation surface 302 covers the cutting electrode 306. As described herein, insulation 308 supports the assembly and electrically separates the cutting electrode 306 from the coagulation electrode 302. Spot welds 310 mechanically secure the layers together.

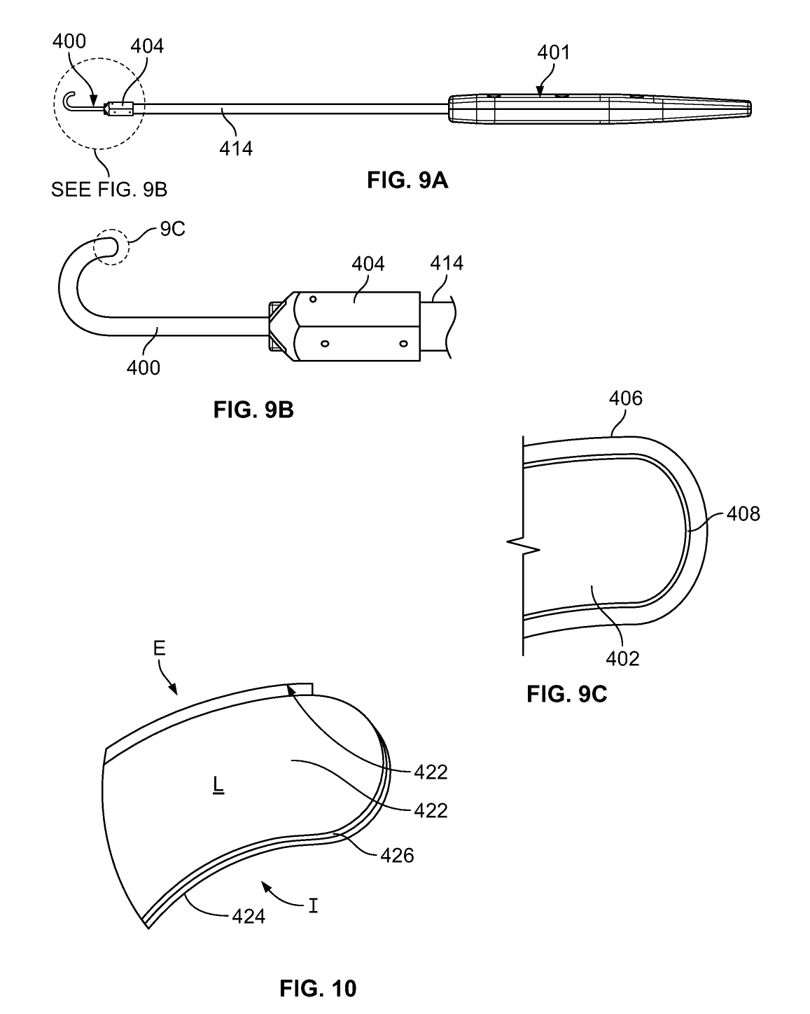

FIGS. 9A-9C show another embodiment including a distal J-hook shaped blade 400, a handle 401, and an elongate shaft 414 extending from the handle to the distal tip 400. A coagulation surface 402 covers the cutting electrode 406. As described herein, insulation 408 supports the assembly. The cutting electrode 406 wraps around the exterior and interior surface or edge of the distal tip.

FIG. 10 is an enlarged view of an alternative variation of the distal tip section shown in FIG. 9C in which a coagulation electrode 422 is present on both the exterior (E) and lateral (L) surfaces. The cutting electrode 424 is only exposed on the interior (I) surface. In a sense, the device is a one sided cutting J-hook. Insulation layer 426 supports the assembly as described herein.

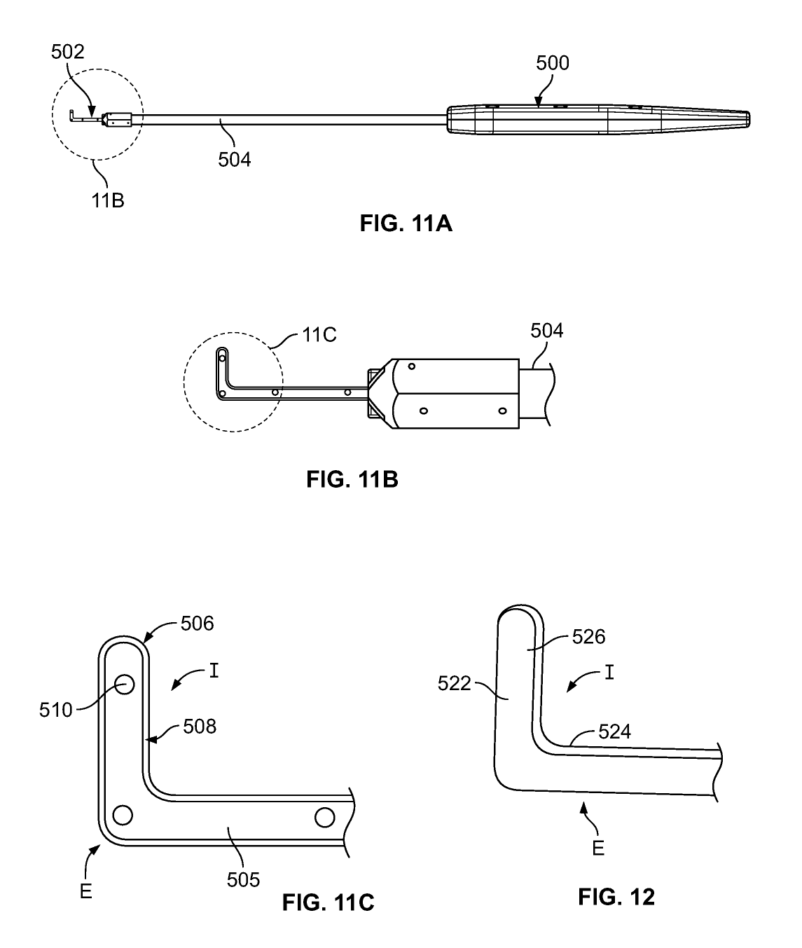

FIGS. 11A-11C depict another electrosurgical handpiece including a distal L-hook shaped blade 502, a handle 500, and an elongate shaft 504 extending from the handle to the distal tip 502. A coagulation surface 505 covers the cutting electrode 506. Insulation 508 supports the assembly. Several weld spots 510 mechanically secure the assembly together without the need for adhesives or spray or dip coatings. However, in some embodiments, an adhesive, spray coating, heat bond, or fusion is applied to the cutting electrode, coagulation electrode, or a combination of the electrodes to form or hold the assembly together. With reference to FIG. 11C, the cutting electrode 506 is shown along the exterior (E) and interior (I) surfaces of the distal tip.

FIG. 12 is an enlarged view of an alternative variation of the distal tip section shown in FIG. 11C in which the cutting electrode 524 is only exposed on the interior (I) surface. In a sense, the device is a one sided cutting L-hook. A large surface coagulation electrode 522 covers the cutting electrode 524. Insulation layer 526 supports the assembly as described herein.

The L and J-hook shaped devices shown and described in FIGS. 9-12 are useful in various procedures including but not limited to laparoscopic procedures. J or L-hooks are exemplary shapes wherein the interior of the hook portion is adapted for cutting and the exterior of the device is used for coagulation.

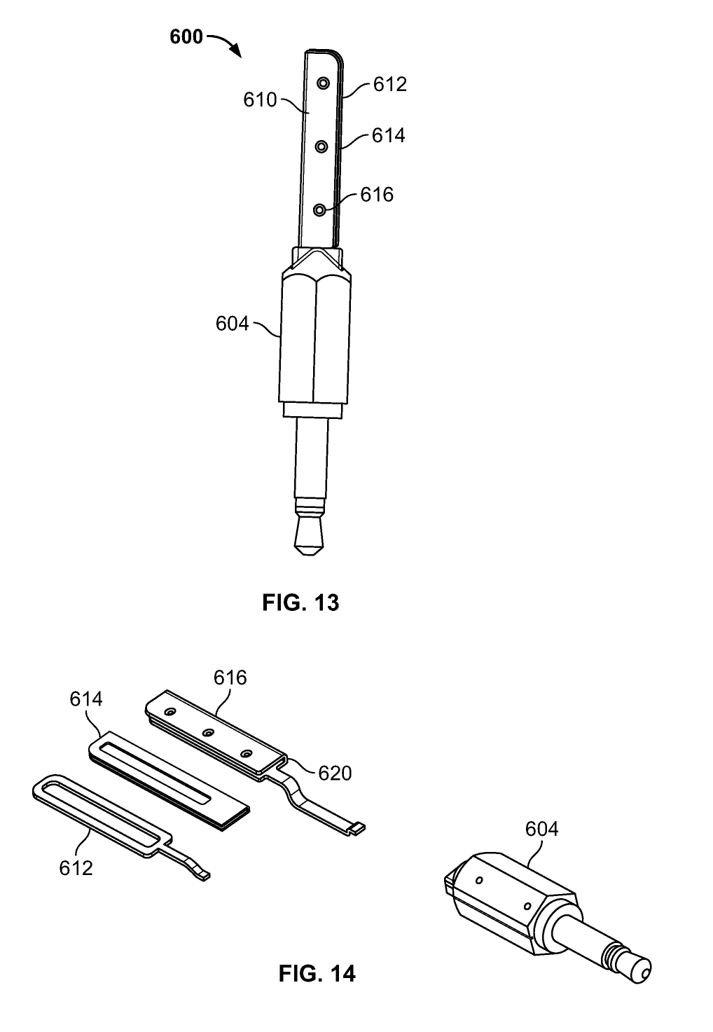

FIGS. 13-14 are various views of another variation of a monopolar-based electrosurgical implement 600 including a tip-support 604 and a distal tip section 610. The implement is similar to that shown in previous described embodiments except the distal tip section is arranged in a folded or taco-like assembly.

With reference to FIG. 14, an exploded view of the components is shown including cutting electrode sheet 612, folded insulating member 614, and folded coagulation electrode sheet 616. Spot welds 616 are applied to fasten the components together. Unlike the embodiment shown in FIG. 5, described above, the distal tip shown in FIG. 13 includes a spine or joint portion 620 which connects one side of the coagulation electrode to an opposing side of the coagulation electrode. Consequently, an intermediate electrically conducting bridge such as the intermediate electrically conducting piece 86 described in FIG. 5 is unnecessary in the distal tip shown in FIG. 13.

Although a number of embodiments have been disclosed above, it is to be understood that other modifications and variations can be made to the disclosed embodiments without departing from the subject invention.

1. A monopolar-based electrosurgical handpiece, the handpiece operable with a generator and a first active lead and a remote return electrode both coupled to the generator, the handpiece comprising:
a body, and a distal tip;
a first electrode located in the distal tip, and coupleable to the first active lead for cutting tissue, together with the remote return electrode, the first electrode comprising a discrete exposed cutting surface area;
a second electrode located in the distal tip, and coupleable to the first active lead for coagulating tissue, together with the remote return electrode, the second electrode comprising a discrete exposed coagulating surface area larger than the discrete exposed cutting surface area;
an electrically non-conducting support secured to the first electrode, and defining the discrete exposed cutting surface area where the first electrode is uncovered by the electrically non-conducting support, and electrically separating the first electrode from the second electrode; and
at least one switch in the body for selectively coupling the first active lead to one of the first electrode and the second electrode.

2. The apparatus of claim 1 wherein the first electrode is thin and planar shaped.

3. The apparatus of claim 2 wherein the second electrode comprises two thin and planar-shaped coagulation members.

4. The apparatus of claim 3 wherein the two coagulation members sandwich the first electrode therebetween.

5. The apparatus of claim 4 wherein the two coagulation members are electrically connected and mechanically affixed to one another through an aperture in the first electrode.

6. The apparatus of claim 5 wherein the first electrode has a uniform thickness.

7. The apparatus of claim 6 wherein the thickness of the first electrode ranges from 0.002 to 0.010 inches.

8. The apparatus of claim 1 wherein substantially all of the first electrode is covered by the electrically non-conducting body.

9. The apparatus of claim 1 wherein the electrically non-conducting support is mechanically secured between the first electrode and the second electrode.

10. The apparatus of claim 9 wherein the electrically non-conducting support is mechanically secured between the first electrode and the second electrode by use of one or more spot welds or fasteners.

11. The apparatus of claim 9 wherein the electrically non-conducting support is affixed adhesive-free.

12. The apparatus of claim 1 wherein the electrically non-conducting support is a material selected from fluoropolymer or ceramic.

13. The apparatus of claim 1 wherein the thickness of the electrically non-conducting support ranges from 0.001 to 0.010 inches.

14. The apparatus of claim 1 wherein the electrically non-conducting support comprises a plurality of discrete layers.

15. The apparatus of claim 5 wherein each of the two coagulation members has a thickness ranging from 0.005 to 0.020 inches.

16. The apparatus of claim 1 further comprising a tip-support housing joining the distal tip to the body and wherein tip-support housing is detachably joined to the body.

17. The apparatus of claim 1 wherein the body comprises at least one actuating element to operate the at least one switch selected from the group consisting of button, lever, knob, slide, and trigger.

18. The apparatus of claim 1 wherein the distal tip has a hook shape.

19. The apparatus of claim 1 further comprising a third electrode located in the distal tip, and coupleable to the first active lead for coagulating tissue, together with the remote return electrode.

20. The apparatus of claim 1 wherein the coagulating surface area is at least 4 times larger than the cutting surface area.

21. The apparatus of claim 1 wherein the at least one switch is configured to send a coagulation signal to the first and the second electrode when the generator is operable in the coagulation mode.

22. The apparatus of claim 1 wherein the distal tip is operable in dry-field applications.

23. A monopolar-based electrosurgical system for cutting and coagulating tissue, the system comprising:
a generator operable in a cutting mode and a coagulation mode, and a first active lead and a remote return electrode coupled to the generator; and
a handpiece, the handpiece comprising:
a body, and a distal tip;
a first electrode located in the distal tip, and coupleable to the first active lead for cutting tissue, together with the remote return electrode when the generator is operated in the cutting mode, the first electrode comprising a discrete exposed cutting surface area;
a second electrode located in the distal tip, and coupleable to the first active lead for coagulating tissue, together with the remote return electrode when the generator is operated in the coagulation mode, the second electrode comprising a discrete exposed coagulating surface area larger than the discrete exposed cutting surface area;
an insulator mounted between the first electrode and second electrode, and the insulator covering the first electrode to define the discrete exposed cutting surface area where the first electrode is uncovered by the insulator, and to electrically separate the first electrode from the second electrode; and
at least one control feature in the body for selectively coupling the first active lead to one of the first electrode and the second electrode corresponding to the cutting mode and coagulation mode respectively.

24. The system of claim 23 wherein the control feature is a switch configured to send a coagulation signal solely to the second electrode when the generator is operable in the coagulation mode.

25. The system of claim 23 wherein the control feature is a one switch configured to send a coagulation signal to the first and the second electrode when the generator is operable in the coagulation mode.

26. A method of manufacturing a monopolar-based electrosurgical handpiece for use with a generator having a cutting mode and a coagulation mode, the method comprising:
arranging an elongate planar-shaped electrically non-conducting body on each side of an elongate planar-shaped cutting electrode, and an elongate planar-shaped coagulation electrode on top of each elongate planar-shaped electrically non-conducting body such that a cutting surface area of the cutting electrode is left exposed; and
mechanically securing each coagulation electrode and electrically non-conducting body to the cutting electrode.

27. The method of claim 26 wherein the step of mechanically securing is performed by welding.

28. The method of claim 26 wherein each elongate planar-shaped electrically non-conducting body comprises an opening extending there though.

29. The method of claim 28 wherein the elongate planar-shaped cutting electrode comprises an aperture extending there though, and wherein each opening in the electrically non-conducting body is aligned with the aperture.

30. The method of claim 29 further comprising positioning within each said opening and aperture an elongate planar-shaped electrically conducting support, the support electrically connecting each elongate planar-shaped coagulation electrode to form an integrated multi-surface coagulation electrode.

31. The method of claim 30 wherein the elongate planar-shaped electrically conducting support and elongate planar-shaped coagulation electrode are stainless steel.

32. A monopolar-based electrosurgical implement, the implement operable with a handpiece, generator and a first active lead and a remote return electrode both coupled to the generator, the implement comprising:
a thin, planar-shaped distal tip, the distal tip comprising
a first electrode, and coupleable to the first active lead for cutting tissue, together with the remote return electrode, the first electrode comprising a discrete exposed cutting surface area;
a second electrode, and coupleable to the first active lead for coagulating tissue, together with the remote return electrode, the second electrode comprising a discrete exposed coagulating surface area larger than the discrete exposed cutting surface area; and
an electrically non-conducting support secured to the first electrode, and defining the discrete exposed cutting surface area where the first electrode is uncovered by the electrically non-conducting support, and electrically separating the first electrode from the second electrode;
a tip-support housing from which the distal tip extends distally; and
an electrical connector adapted to electrically couple with the handpiece.

33. The monopolar-based electrosurgical implement of claim 32 wherein the connector is a pin extending proximally from the tip-support housing.
